(19) United States
(12) Patent Application Publication
(10) Pub. No.: US 2021/0395766 A1
(43) Pub. Date: Dec. 23, 2021

(54) METHOD FOR ENZYMATICALLY MODIFYING THE TRI-DIMENSIONAL STRUCTURE OF A PROTEIN

(71) Applicant: Luxembourg Institute of Science and Technology (LIST), Esch-sur-Alzette (LU)

(72) Inventor: Kjell Sergeant, Vauxsur-Sure (BE)

(21) Appl. No.: 17/388,190

(22) Filed: Jul. 29, 2021

Related U.S. Application Data
(63) Continuation-in-part of application No. 16/062,198, filed on Jun. 14, 2018.

(30) Foreign Application Priority Data
Dec. 14, 2015 (LU) 92906

(51) Int. Cl. C12N 15/82; C12N 9/20; C07K 14/62

(52) U.S. Cl. CPC C12N 15/8251; C07K 14/62; C12Y 301/01003; C12N 9/20

(57) ABSTRACT

A structurally-modified recombinant protein, obtained by a method comprising generating at least one genetic construct comprising a nucleotide sequence coding for the protein comprising a recognition sequence; expressing in a host the at least one genetic construct using a vector comprising the at least one genetic construct; and using a plant-based expression system with the vector to express the protein, the plant-based expression system being a plant or a plant cells suspension; the recognition sequence comprises a sequence Phe-x1-x2-Tyr, wherein Phe is phenylalanine, x1 and x2 are amino acid residues, and Tyr is tyrosine and the plant-based expression system has an inherent enzymatic activity which converts the phenylalanine residue of the recognition sequence into a didehydrophenylalanine residue, producing a structurally-modified recombinant protein; and isolating the protein with the recognition sequence which is a part of the protein, the phenylalanine being converted to a didehydrophenylalanine from the plant-based expression system.

### CROSS-REFERENCE TO RELATED APPLICATIONS

This application is a continuation-in-part of U.S. patent application Ser. No. 16/062,198 filed on Jun. 14, 2018, which is a US national stage under 35 U.S.C. § 371 of International Application No. PCT/EP2016/064094, which was filed on Jun. 17, 2016, and which claims the priority of application LU 92906 filed on Dec. 14, 2015, the content of which (text, drawings and claims) are incorporated here by reference in its entirety.

### SEQUENCE LISTING STATEMENT

The official copy of the sequence listing is submitted electronically via EFS-Web as an ASCII formatted sequence listing with a file named “Sequence Listing_Amend_3_LEPA_F351W1”, created on Mar. 26, 2020, and having a size of “8 KB”. The sequence listing contained in this ASCII formatted document is part of the specification and is herein incorporated by reference in its entirety.

### FIELD

The invention is directed to the field of stabilization of proteins in order to enhance their specificities and their activities. For example, in various exemplary embodiments the invention is directed to the incorporation and use of modified amino acid residues in order to stabilize the structure of proteins. For example, the modified amino acid residue is didehydro-phenylalanine.

### BACKGROUND

Proteins are remarkably dynamic macromolecules, with conformational motions that play roles in all biological processes such as generating mechanical support, carrying out enzymatic reactions, and mediating signal transduction. Since the various states, more particularly the conformation of a protein molecule, may potentiate different functions there is considerable interest in the ability to generate proteins with a stable and predictable three dimensional structure.

The use of enzymes in industry and of proteins in general is still restricted in many cases. The greatest technical difficulty is the finding of suitable proteins which are stable under industrially desired conditions such as temperature, pH, requirements of activators, and/or the presence of inhibitors. The use of enzymatic reactions in the catalysis during industrial applications is thus limited by insufficient stability of the enzymes under the used reaction conditions or during purification.

Similarly, the use of protein therapeutics is hindered by the limited stability during long-term storage of the products.

US patent application published US 2013/281314 A1 relates to methods for screening for and using conformationally stabilized forms of a conformationally dynamic protein, such as a conformationally stabilized ubiquitin protein.

International patent application published WO 2011/073209 A1 refers to novel proteins, in particular hetero-multimeric proteins, capable of binding the extradomain B of fibronectin (ED-B). Furthermore, the disclosure refers to fusion proteins comprising the binding protein fused to a pharmaceutically and/or diagnostically active component. The disclosure is further directed to a method for the generation of such binding protein or fusion protein and to pharmaceutical/diagnostic compositions containing the same. In addition, the disclosure refers to libraries which are based on a scaffold protein comprising linear polyubiquitin chains with at least two interacting binding determining regions (BDR).

International patent application published WO 01/29247 A1 relates to cross-linking methods to stabilize polypeptides and polypeptide complexes for commercial uses (pharmaceutical, therapeutic, and industrial).

Several protein stabilization strategies are known in the art and are briefly reviewed here.

On the protein level, the most prominent approach is the discovery of stable biocatalysts from extremophilic and more particularly thermophilic, organisms, directed evolution, and computational and protein engineering. As most industrial enzymes are preferably used at elevated temperatures so that viscosity is reduced while reaction rates are increased industrial enzymes are often best derived from thermophilic microorganisms or extremophiles which resist to these extreme conditions.

For example, U.S. Pat. No. 8,592,192 relates to the field of stabilizing proteins, and more specifically to the field of stabilizing proteins without any modification of their primary sequence. Although enzymes of commercial relevance have been identified from them, this ‘discovery’ approach is limited by what can be found in nature. This approach has not yielded many commercially-relevant, thermostable biocatalysts as was initially hoped for and/or projected.

‘Directed evolution’ techniques are powerful approaches capable of generating stabilized enzymes, often also with altered/improved functional specificities. However, the approach is limited by the feasibility of the selection procedure.

Algorithms that calculate intra-molecular forces within proteins are being used to design and/or evolve enzymes with greater thermostability in silico. This approach is still severely hampered by the limited understanding of the intra-molecular forces and the processes involved in protein folding.

US patent application published US 2014/012777 relates to improved stabilization of polypeptides by incorporation of non-natural amino acids, such as hyper-hydrophobic amino acids, into the hydrophobic core regions of the polypeptides.

International patent application published WO 2008/085900 A2 relates to biomolecular engineering and design, including methods for the design and engineering of biopolymers such as proteins and nucleic acids.

U.S. Pat. No. 5,811,515 generally relates to the synthesis of conformationally restricted amino acids and peptides. More specifically, the invention relates to the synthesis of conformationally restricted amino acids and peptides by catalyzed ring closing metathesis (“RCM”). Other approaches, often referred to as protein engineering, such as derivatization (e.g. PEGylation, addition of polymeric sucrose and/or dextran, methoxypolyethylene glycol, etc.) and old methods of protein cross-linking (e.g. production of cross-linked enzyme crystals or CLEC's) can also be cited. Unfortunately, these approaches are often ineffectual or cause dramatic losses in activity.

European patent application EP 0355 039 A2 is linked to the field of protein engineering and provides methods for the production of proteins with modified stability, preferably towards thermal denaturation and/or chemical modification, by means of one or more amino acid replacements at specific sites in proteins using protein engineering techniques.

Other strategies, such as (a) catalyst immobilization or (b) use of organic solvents in the reaction medium (termed medium engineering) have been employed.

However, despite the great technological potential of catalyst immobilization, few large-scale processes utilize immobilized enzymes. Severe restrictions often arise in scale-up because of additional costs, activity losses, and issues regarding diffusion.

Regarding the medium engineering field, enhanced thermostability in organic media has proven to be an additional and significant bonus. It is hypothesized that partial or almost total substitution of water can be beneficial since water is involved in enzyme inactivation. Whatever the mechanism, numerous cases have recently been reported where remarkable enzyme stability has been obtained in organic media such as polyglycols and glymes. However, medium engineering is unlikely to solve all biocatalysis stability problems.

Molecular biological techniques have made it possible to stabilize some proteins by, e.g., engineering fusion-proteins. To make a fusion-protein, a single nucleic acid construct is created that directs the expression of modular domains derived from at least two proteins as one protein. Due to fusion, the two domains are held in very close proximity to each other, one keeping the other stable and in solution (Harada et al., Cancer Res., 2002, 62, 2013-2018).

However, in the design of pharmacological reagents, it is often disadvantageous to create fusion proteins that require a linker sequence to stabilize them.

Australian patent application published AU 2008 202 293 A1 relates to methods of introducing one or more cysteine residues into a polypeptide which permit the stabilization of the polypeptide by formation of a disulfide bond between different domains of the polypeptide. The disclosure also relates to polypeptides containing such introduced cysteine residue(s), nucleic acids encoding such polypeptides and pharmaceutical compositions comprising such polypeptides or nucleic acids

Disulfide bonds are, however, unstable under many physiological conditions. Physiological conditions vary widely, for instance with respect to redox potential (oxidizing vs. reducing) and acidity (high vs. low pH) of the various physiological milieus (intracellular, extracellular, pinocytosis vesicles, gastro-intestinal lumen, etc.). Disulfide bonds are known to break in reducing environments, such as the intracellular milieu. But even in the extracellular milieu, engineered disulfide bonds are often unstable.

Several other chemical cross-link methodologies allow the formation of bonds that are stable under a broad range of physiological and non-physiological pH and redox conditions. However, in order to maintain the complex's activity and specificity, it is necessary that the cross-link is specifically directed and controlled such that, first, the overall structure of the protein is minimally disrupted, and second, that the cross-link is buried in the protein complex so as not to be immunogenic. However with most cross-link methodologies, the degree to which a bond can be directed to a specific site is too limited to allow them to be used for most bio-pharmaceutical and/or diagnostic applications.

Examples of such cross-link methodologies include UV-cross-linking, and treatment of protein with formamide or glutaraldehyde.

Immunoglobulin Fv fragments comprise another example of a class of proteins for which stabilization is desirable. Immunoglobulin Fv fragments are the smallest fragments of immunoglobulin complexes shown to bind antigen. Fv fragments consist of the variable regions of immunoglobulin heavy and light chains and have broad applicability in pharmaceutical and industrial settings.

To date, a variety of methodologies have been employed to stabilize engineered antibodies. First, introduction of additional disulfide bonds has been performed through molecular biological manipulation of the antibody-expressing construct, without however resolving all the above mentioned drawbacks regarding the use of disulfide bonds. Second, introduction of a linker has been employed that allows both fragments to be expressed as a single chain. Yet, linkers result in rigid conjugates that elicit immune responses, hampering the utility. Linkers that are not immunogenic are generally the more flexible linkers that provide insufficient stability. Finally, fusion of an exogenous di- or oligomerization domain to each of the Fv fragment chains has been performed. Unfortunately, it appears that Fv fragments stabilized by fusion to multimerization domains are significantly immunogenic, and lack the most significant advantage of Fv fragments in the first place: reduced size and resultant increased tissue penetration.

Another approach could be the oxidative cross-link reaction between tyrosyl side-chains, which has been demonstrated to occur naturally, for example in the cytochrome c peroxidase compound I.

The reaction only occurs with tyrosine side-chains that are in very close proximity to each other. Furthermore, the bond formed between the tyrosyl side-chains is irreversible and stable under a very wide range of physiological conditions. Furthermore, the use of dityrosyl cross-linking for formation of buried chemical cross-links for stabilizing a protein complex while maintaining its activities and specificities have not been described in a commercial setting.

International patent application published WO 2015/013551 A1 describes constructs to stabilize or “lock” the respiratory syncytial virus (RSV) F protein in its pre-fusion conformation. The RSV F protein is known to induce potent neutralizing antibodies that correlate with protection against the virus. This disclosure provides RSV F polypeptides, proteins, and protein complexes, such as those that can be or are stabilized or “locked” in a pre-fusion conformation, for example using targeted cross-links, such as targeted di-tyrosine cross-links. This disclosure also provides specific locations within the amino acid sequence of the RSV F protein at which, or between which, cross-links can be made in order to stabilize the RSV F protein in its pre-F conformation. Where di-tyrosine crosslinks are used, the disclosure provides specific amino acid residues (or pairs of amino acid residues) that either comprise a pre-existing tyrosine residue or can be or are mutated to a tyrosine residue such that di-tyrosine cross-links can be made.

In the search to produce proteins with an increased stability, researchers have demonstrated in US patent application published US 2012/0141423 A1 that modified residues comprising notably dehydrated amino acids, i.e., α,β-didehydroalanine (Dha) and α,β-didehydrobutyric acid (Dhb) and thioether bridges of the nonproteinogenic amino acid lanthionine, can stabilize molecular conformations that are essential for the antimicrobial activity of antimicrobial peptide (AMP). An example of AMP which is stabilized with dehydrated amino acids residue is nisin, a lantibiotic approved by the World Health Organization as a food preservative. Other works concerning lantibiotic and stabilizing dehydrated amino acids are described in U.S. Pat. No. 5,932,469 which relates to bacteriocins, in U.S. Pat. No. 7,479,781 B2 which relates to compounds and pharmaceutical compositions for the treatment of ocular diseases and disorders, or in U.S. Pat. No. 8,691,773 B2 which relates to a peptide compound with biological activity, in particular possessing antimicrobial properties.

Didehydro-phenylalanine (Phe) is regarded as being among the best choices to fix the 3D structure of short, often non-ribosomal peptides. However the potential to introduce ΔPhe in proteins produced using a natural production system was previously unknown, hence ΔPhe could only be introduced in intact polypeptides using solid phase protein synthesis or similar chemical techniques as was demonstrated by the introduction of a functional hinge in insulin (Menting et al. PNAS, 2014, E3395-E3404), a production system unsuitable for large-scale production of catalysts for industrial applications.

### SUMMARY

The invention has for technical problem to alleviate at least one of the drawbacks present in the prior art.

An object of the invention is generically directed to a method for incorporating a recognition sequence in a protein, the method comprising the steps of (a) generating at least one genetic construct comprising the recognition sequence; (b) expressing in a host the at least one genetic construct using oligonucleotide primers, thereby forming a vector; and (c) using a plant cell-based expression system with a constitutive or inducible promoter to express the vector. The method is remarkable in that the recognition sequence comprises the sequence Phe-x1-x2-Tyr, wherein Phe is phenylalanine, x1 and x2 are amino acid residues and Tyr is tyrosine.

One aspect of the invention relates to a structurally-modified recombinant protein, obtained by a method for producing the structurally-modified recombinant protein, comprising the steps of: (a) generating at least one genetic construct comprising a nucleotide sequence coding for the protein comprising a recognition sequence; (b) expressing in a host the at least one genetic construct using a vector comprising the at least one genetic construct; and using a plant-based expression system with the vector to express the protein, the plant-based expression system being a plant or a plant cells suspension; the recognition sequence comprises a sequence Phe-x1-x2-Tyr, wherein Phe is phenylalanine, x1 and x2 are amino acid residues, and Tyr is tyrosine and the plant-based expression system has an inherent enzymatic activity which converts the phenylalanine residue of the recognition sequence into a didehydrophenylalanine residue, resulting in the structurally-modified recombinant protein, and at least one subsequent step of (c) isolating the protein with the recognition sequence which is a part of the protein, wherein the phenylalanine has been converted to a didehydrophenylalanine from the plant-based expression system.

In an exemplary embodiment, x1 and x2 may be polar hydroxyl-containing amino acids and/or basic amino acids. Significantly overrepresented among amino acids as x1 and x2 in the recognition sequence may advantageously be the hydroxyl-containing amino acids Thr and Ser, the basic amino acids Lys and Arg and the amide Asn, together accounting of from 70% to 80%, in various instances of from 70% to 76%, in some embodiments of from75% to 80%, or even 76% of the amino acids found at the positions x1 and x2 of the sequence Phe-x1-x2-Tyr.

In the context of the invention, the sequence Phe-x1-x2-Tyr is defined with

Phe being the amino acid phenylalanine at position 1 and Tyr, being the amino acid tyrosine at position 4. At the position 2 defined as x1, the amino acids Thr and Lys are in various instances and at the position 3 defined as x2 the amino acids Ser, Thr and Asn are preferred example. In an exemplary embodiment, aromatic (Tyr and Phe), branched chain hydrophobic (Ile, Leu and Val) and acidic amino acids may rarely be found in the x1 and x2 position (≤2% occurrence), while Cys, Pro and Trp may be absent at these positions.

In step c), isolating the protein with the recognition sequence is a part of the protein, wherein the phenylalanine has been converted to a didehydro-phenylalanine from the plant-based expression system can be understood as isolating the protein with the recognition sequence with a didehydro-phenylalanine residue.

In various embodiments, the structurally-modified protein is used as a biocatalyst, or as a protein therapeutic.

In an exemplary embodiment, the plant cell-based expression system is based on at least one plant belonging to the clades of rosids, preferentially to the clades of fabids or malvids.

In an exemplary embodiment, the plant-based expression system is based on Medicago sativa, Arabidopsis thaliana and/or Cannabis sativa.

In an exemplary embodiment, the at least one genetic construct comprising the recognition sequence may be based on a focus sequence of one beta subunit of polygalacturonase of Medicago sativa represented by SEQ-ID NO:3-6.

In the context of the invention, “residue” means one of the 20 proteaginous amino acids.

In the context of the invention, “host” is defined as the plant or plant cell used to express the recombinant protein containing the Phe-x1-x2-Tyr recognition sequence.

In the context of the invention, “vector” is defined as a genetic construct used to express the recombinant protein containing the sequence Phe-x1-x2-Tyr in a plant or plant cell system.

In the context of the invention, “enzymatic activity” is defined as the enzymatic activity inherently present in all plants that converts Phe in the sequence Phe-x1-x2-Tyr to didehydrophenylalanine.

According to the invention, a plant may be any species classified as being part of the kingdom Plantae. A plant cell suspension is a suspension made of cell(s) isolated from any species part of the taxonomical kingdom Plantae.

According to the invention, the structurally-modified recombinant proteins, like insulin or lipase, are characterized by the presence of the sequence Phe-x1l-x2-Tyr with the Phe in this sequence converted to didehydrophenylalanine, the structure modification of this recombinant protein compared to the non-modified ensues from steric restraints of didehydrophenylalanine as is common scientific knowledge (Crisma et al. J. Am. Chem. Soc. 1999, 121, 14, 3272-3278 https://doi.org/10.1021/ja9842114).

The second object of the present invention is directed to a method for producing a structurally-modified protein, comprising the method in accordance with the first object of the invention and at least one subsequent step of isolation from the plant cell-based expression system.

The third object of the present invention is directed to a structurally-modified protein obtainable by the method in accordance of the second object of the present invention.

The structurally-modified recombinant proteins may advantageously be at least one of lipases, proteases and nucleotide ligases.

Lipases are a subclass of the esterases. Lipases perform essential roles in digestion, transport and processing of dietary lipids (e.g. triglycerides, fats, oils) in most, if not all, living organisms. Genes encoding lipases are even present in certain viruses.

Most lipases act at a specific position on the glycerol backbone of a lipid substrate (A1, A2 or A3) (small intestine). For example, human pancreatic lipase (HPL), which is the main enzyme that breaks down dietary fats in the human, converts triglyceride substrates found in ingested oils to monoglycerides and two fatty acids.

Proteases may be at least one among Serine proteases, Cysteine proteases , Threonine proteases, Aspartic proteases, Glutamic proteases, Metalloproteases and Asparagine peptide lyases.

Nucleotide ligase are ligases that join DNA strands through the formation of a phosphodiester bond, performing essential steps in the formation of recombinant polynucleotides widely used in molecular biology and biotechnology applications.

As an example, the structurally-modified recombinant protein may be lipase or insulin.

The enzymatic conversion of a normal phenylalanine residue to a didehydro-form uses the enzymatic machinery inherently present in plant cells or plants that coverts Phe in the sequence Phe-x1-x2-Tyr into didehydrophenylalanine, thereby generating recombinant proteins with the desired, stable fold. Compared to other solutions for the technical problem, it avoids the low yields for chemically introducing unnatural amino acids in recombinant proteins (and associated high production cost). In Menting et al. (PNAS 2014, 111(33) E3395-E3404) chemical peptide synthesis was used to synthesize insulin analogues ΔPhe^{B25} and ΔPhe^{B24} with a didehydrophenylalanine replacing the Phe at the positions 25 and 24 of the insulin beta chain. These replacements of Phe by ΔPhe change the receptor binding of the insulin, thereby providing rationalization for designing new therapeutic insulin analogs. While chemical peptide synthesis is feasible and a useful tool for research purposes, the cost is prohibitively high for producing modified proteins for therapeutic or biocatalysis use. In other words, the technical problem may be therefore to allow the smooth insertion of didehydro-phenylalanine into a recombinant protein being in order to stabilize the protein. The recognition sequence has not been chosen arbitrarily because this is a sequence which is recognized with high precision by the enzymatic system present in the expression system.

### DRAWINGS

FIG. 1 is an exemplary representation of the MS/MS spectrum of the peptide represented by SEQ-ID NO:1.

FIG. 2 is an exemplary table comprising the peaks of the MS/MS spectrum of FIG. 1.

FIG. 3 exemplarily shows the bioinformatic analysis of 512 recognition sequences from βPG proteins from plant species covering the entire kingdom Plantae. These sequences are obtained from the NCBI database and the analysis and graphical representation is generated with WebLOGO (https://weblogo.berkeley.edu/logo.cgi). The position 1 is Phe while at 4 Tyr makes up for 100%. The dominance of Thr, Ser, Lys, Arg and Asn at the positions 2 and 3, respectively being x1 and x2 in the annotation Phe-x1-x2-Tyr is shown.

### DETAILED DESCRIPTION

The invention proposes the incorporation of the sequence Phe-x1-x2-Tyr, at critical positions in recombinant proteins, a phenylalanine that after enzymatic modification provides a conformationally-restrained bending point in the 3D structure of the protein.

The sequence Phe-x1-x2-Tyr, hereafter defined as recognition sequence, provides a sequence targeted by an enzymatic activity inherently present in plant cells that converts the Phe of the sequence to the structure defining amino acid derivative didehydrophenylalanine, according to the description given here below.

The beta subunit of polygalacturonase (βPG), the recognition sequence Phe-x1-x2-Tyr and the formation of didehydrophenylalanine from Phe in this recognition sequence is based on current scientific knowledge universal in all organisms in the taxonomical kingdom “plantae”.

βPG is part of a plant-specific group of proteins that contain the plant-specific BURP-domain (NCBI conserved domain database entry c103923) at their C-terminus (Hattori, J et al. A conserved BURP domain defines a novel group of plant proteins with unusual primary structures. Mol Gen Genet 259, 424-428 (1998). https://doi.org/10.1007/s004380050832).

A gene coding for βPG is found in all sequenced plant genomes.

βPG is synthesized as a 3-domain precursor: a N-terminal domain containing a signal-and pro-peptide, a central domain composed of repeats of 14 amino acids starting with the sequence Phe-x1-x2-Tyr and a C-terminal BURP domain of unknown function but essential for phenotype effects (Park J et al. AtPGL3 is an Arabidopsis BURP domain protein that is localized to the cell wall and promotes cell enlargement. Front Plant Sci. 2015; 6: 412. doi:10.3389/fpls.2015.00412). Bioinformatic analysis of current sequence databases shows that only in βPG from plants domains composed of repeated sequences starting with Phe-x1-x2-Tyr are found.

In the plant cell wall, the subcellular location where βPG has its physiological function, only the central domain is found, thus forming the active protein. Amino acid analysis of this active protein indicates that of the expected 23 Phe residues, based on the genome sequence, only 2 are found. For all other amino acids the number expected based on the genome sequence is found. Furthermore peptide sequencing using Edman-degradation returns a blank cycle when a Phe residue is expected based on the genome sequence (Zheng L et al. The beta subunit of tomato fruit polygalacturonase isoenzyme 1: isolation, characterization, and identification of unique structural features. Plant Cell. 1992; 4(9): 1147-1156. doi:10.1105/tpc.4.9.1147). Detailed analysis of the active domain of tomato βPG (NCBI entry Q40161.1 residue 110-412) shows that of the 23 Phe residues expected based on the genome sequence only two are not found in the sequence Phe-x1-x2-Tyr, corresponding to the two Phe residues quantified by amino acid analysis.

Modification of an amino acid changes the chromatographic retention time of its derivatives as generated for identification and quantification respectively in Edman degradation and amino acid analysis. This indicates that all Phe residues in the sequence Phe-x1-x2-Tyr but not Phe residues in other sequences are modified.

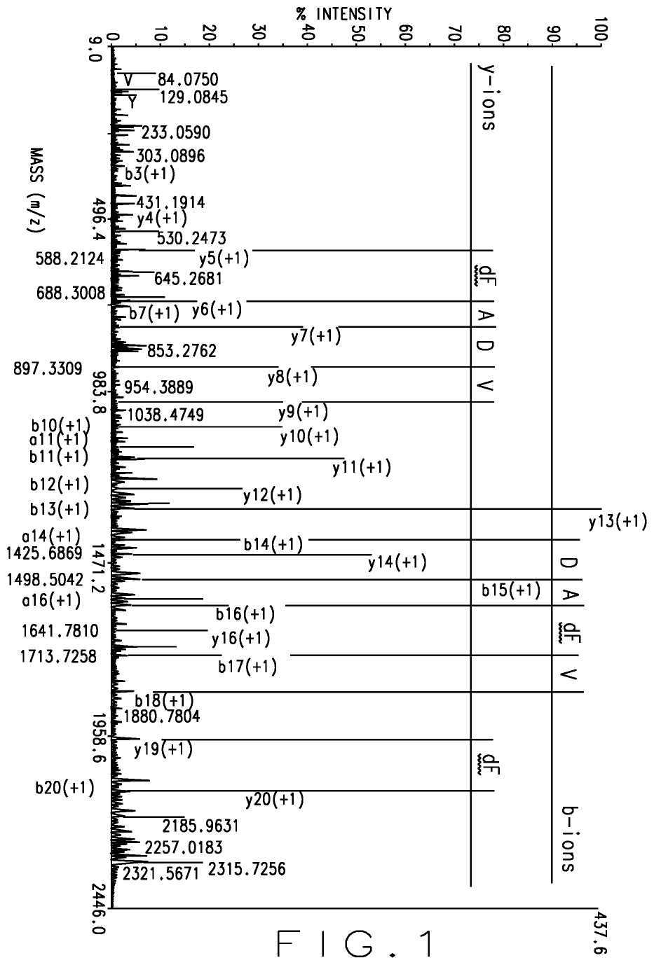

The modification was identified as being the loss of 2 Da from the Phe-residue (FIG. 1) which, based on the chemical structure of Phe, can only be attributed to the formation of a double bond between the alpha- and beta-carbon of Phe, resulting in the formation of didehydrophenylalanine. A modification that causes the change in chromatographic retention time and thus the lack of identification/quantification with respectively Edman degradation and amino acid analysis.

The modification is found in βPG homologous in different plant species (Arabidopsis maize, Cannabis sativa, Medicago sativa) and βPG was never identified without this modification. Together with the omnipresence of βPG in plants, the conservation of its sequence in all plant taxa, the impact of didehydro amino acids on protein fold and thus structure and the fact that functional βPG is essential for plant growth this indicates that Phe-residues in the sequence Phe-x1-x2-Tyr in βPG have the same modification in all plant species.

No proteins homologous to βPG are found outside of plant taxa, not has the modification been found in searches in proteins from organisms that are not classified as plants.

The presence of a rare modification on one specific amino acid of one specific protein is similar to what is known for diphthamide (Liu S et al. Diphthamide modification on eukaryotic elongation factor 2 is needed to assure fidelity of mRNA translation and mouse development Proc Nat Ass Sci 2012, 109 (34), 13817-13822. https://doi.org/10.1073/pnas.1206933109).

Although increased during exposure to stress (Ding X. et al. Genome-wide identification of BURP domain-containing genes in rice reveals a gene family with diverse structures and responses to abiotic stresses. Planta. 2009; 230(1):149-163. doi:10.1007/s00425-009-0929-z), βPG genes are expressed in all stages of plant development (Liu H. et al. Overexpression of stress-inducible OsBURP16, the beta subunit of polygalacturonasel, decreases pectin content and cell adhesion and increases abiotic stress sensitivity in rice. Plant Cell Environ. 2014; 37(5):1144-1158.doi:10.1111/pce.12223). The impact of didehydro amino acids on protein fold and structure and the need for βPG for plant growth indicates that the unknown enzymatic activity is inherently present in all plants at all stages of development, although induced during exposure to stress.

The repeated Phe-x1-x2-Tyr recognition sequences of 100 βPG proteins found in NCBI were analyzed with bioinformatics. Significantly overrepresented among amino acids as x1 and x2 in the recognition sequence are the hydroxyl-containing amino acids Thr and Ser, the basic amino acids Lys and Arg and the amide Asn, together accounting for 76% of the amino acids found at the positions x1 and x2 of the sequence Phe-x1-x2-Tyr. Significantly underrepresented as x1 and x2 amino acids are the aromatic amino acids Phe and Tyr, the branched hydrophobic amino acids Ile, Leu and Val, the acidic amino acids Asp and Glu and the amino acids Met and His. While the small hydrophobic amino acid Gly is found proportionally, the amino acids Cys, Pro and Trp are completely absent from the analyzed Phe-x1-x2-Tyr sequences.

A typical Phe-x1-x2-Tyr recognition sequence is Phe-(Thr/Lys)-(Ser/ Asn)-Tyr but variation in x1 and x2 does not impede the conversion of Phe in the sequence Phe-x1-x2-Tyr to didehydrophenylalanine (see examples SEQ-ID No:1-6).

The conformational determination this invention relates to originates from an enzymatic dehydration of the alpha-beta carbon bond of phenylalanine, Phe in the recognition sequence, by an enzymatic activity inherently present in plant cell based expression systems as detailed in above.

First of all dynamic modelling of the stabilized recombinant protein (hereafter designated as “product”) and variations thereof will be done to identify the molecular form with the highest stability while the enzymatic properties of the product are similar or better than that of the wild type protein. The product has to include the determined recognition sequence, including the phenylalanine residue that is to be dehydrated, for the modifying enzyme.

The recognition sequence consists of the sequence Phe-x1-x2-Tyr, a phenylalanine residue followed by a tyrosine residue, separated by two other residues, i.e. Phe-x1-x2-Tyr with x1 and x2 being amino acid residues, dominantly being polar hydroxyl-containing and/or basic amino acids, as set out above. Given the specificity of the modification to the recognition sequence, both the Phe at position 1 and the tyrosine at the position 4 are essential for the modification to occur.

SEQ-ID NO:1 is part of the protein sequence of the beta subunit of polygalacturanose (alfalfa contig 53863). This particular part of the protein sequence has been identified thanks to mass spectrometry analysis, in particular tandem mass spectrometry (MS/MS). General information about the whole protein sequence can be retrieved on http://plantgrn.noble.org/AGED/.

In SEQ-ID NO:1, the sequences of interest in which the Phe is modified in planta (1) Phe800-Ser-Gly-Tyr803; and (2) Phe814-Val-Ser-Tyr817.

FIG. 1 shows the MS/MS spectrum of peptide represented by SEQ-ID NO:1. The dF residues as indicated on the spectrum corresponds to didehydro-phenylalanine (ΔPhe) with a residual mass of 145 Da compared to the residual mass of 147 Da for the unmodified phenylalanine.

Both recognition sequences (1) and (2) have thus been identified. FIG. 1 specifically indicates the y-ion (i.e., those fragment peaks that appear to extend from the C-terminus) series as well the b-ion (i.e., those fragment peaks that appear to extend from the N-terminus) series.

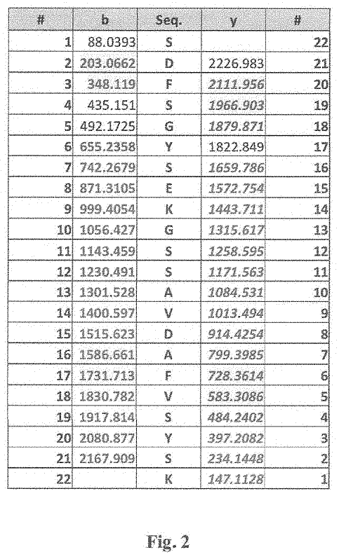

FIG. 2 shows a table corresponding to the matching peaks of the MS/MS spectrum given in FIG. 1. The fragment ions given in b2, b3 and in y20, y19 corresponds to ΔPhe (or dF) from the recognition sequence (1) (1) Phe800-Ser-Gly-Tyr803. The fragments ions given in b16, b17 and in y5, y6 corresponds to ΔPhe (or dF) from the recognition sequence (2) Phe814-Val-Ser-Tyr817. It indeed illustrates the 145 Da mass compared to the mass of 147 Da normally expected for an unmodified Phe.

In order to confirm the results obtained by mass spectrometry, the use of the MASCOT software enables the identification of proteins by interpreting mass spectrometry data.

Searching via MASCOT database thus results in a highly significant match between spectrum and the peptide sequence with ΔPhe. The mascot score is of 148 (a score superior to 47 being considered as significant) and an expected value of 9.3e−0.12.

Using the approach, the presence of the modification of Phe to didehydrophenylalanine when in the sequence Phe-x1-x2-Tyr, was confirmed for the following sequences. SEQ-ID NO:2 gives the completely sequence of the βPG proteins from alfalfa known under the reference alfalfa contig Medtr8g064530. Extracted from this, is the SEQ-ID NO:3 containing the sequence Phe192-Asn-Ser-Tyr195 and Phe206-Lys-Ala-Tyr209 for both of which the Phe was found to be converted to didehydrophenylalanine. SEQ-ID NO:4, SEQ-ID NO:5, and SEQ-ID NO:6 are extracted from the alfalfa contig 53863. SEQ-ID NO:4 contains the sequence Phe102-Thr-Thr-Tyr104, the sequence Phe116-Thr-Ser-Tyr119 and the sequence Phe130-Gly-Asn-Tyr133, the Phe in all the recognition sequences were observed as being dehydrated. Of the 5 Phe-x1-x2-Tyr sequences found in the SEQ-ID NO:5 and SEQ-ID NO:6 4 confirm the dominance of Ser, Thr, Lys and Asn at the x1 and x2 position of the recognition sequence.

The recognition sequence Phe277-Ala-Gly-Tyr280 (SEQ-ID NO:6) does not contain these amino acids in the x1 and/or x2 position but the Phe in this sequence was nonetheless identified as being converted to didehydrophenylalanine. Illustrating that only Phe at position 1 and Tyr at position 4 are essential for the recognition of the Phe at position 1 as amino acid that is converted.

General information about the protein sequence alfalfa contig Medtr8g064530 (SEQ-ID NO:2 and SEQ-ID NO:3) can be retrieved on http://plantgrn.noble.org/Legume|Pv2/.

General information about the protein sequence alfalfa contig 53863 (SEQ-ID NO:4, SEQ-ID NO:5 and SEQ-ID NO:6) can be retrieved on http://plantgrn.noble.org/AGED/.

In order to achieve the introduction of the structure determining modification didehydrophenylalanine into a recombinant protein genetic constructs of the target protein need to be created containing the recognition sequence and expressed in a plant cell-based expression system.

The genetic constructs can be generated using techniques for site-directed mutagenesis. These include classical genetic modification through molecular techniques (Mardanovy et al. Efficient Transient Expression of Recombinant Proteins in Plants by the Novel pEff Vector Based on the Genome of Potato Virus X. Front Plant Sci. 2017, 8, 247. Doi: 10.3389/fpls.2017.00247). A similar approach allows the generation of synthetic genes containing the recognition sequence (Jaynes et al. Plant protein improvement by genetic engineering: use of synthetic genes. Trends Biotech, 1986, 4(12), 314-320. Doi: 10.1016/0167-7799(86)90183-6). Current state-of-the-art genome-editing approaches such as the CRISPR/Cas9 system likewise allow to generate recombinant proteins containing the recognition sequence through insertion into a gene of a nucleotide sequence coding for the recognition sequence (Ma X. et al. CRISPR/Cas9 platforms for genome editing in plants: Developments and applications. Mol Plant, 2016, 9(7) 961-974. Doi: 10.1016/j.molp.2016.04.009).

The thus generated genetic constructs coding for recombinant proteins having the recognition sequence Phe-x1-x2-tyr in their sequence are expressed in plant cell-based expression system (i.e., plant, plant tissues and/or plant cell cultures) using a constitutive or inducible promotor.

Since the modifying enzyme that converts phenylalanine into didehydrophenylalanine is inherently active in the plant cell-based expression system, the Phe in the sequence Phe-x1-x2-Tyr of recombinant proteins containing this recognition sequence is converted into didehydrophenylalanine.

The modifying enzyme converts phenylalanine into didehydro-phenylalanine, thereby determining the tri-dimensional structure of the recombinant protein, stabilizing the protein fold and making it less sensitive to changes of the environment, such as temperature, pH, composition of the solvent, as encountered during isolation, storage and use of recombinant proteins.

The product, i.e. the structurally determined protein containing the recognition sequence and with a didehydrophenylalanine instead of Phe at the first position of the recognition sequence, can be isolated from the culture matrix (plants, plant tissue culture or plant cell cultures) using a pull-down approach with antibodies or other techniques currently used in the art to isolate recombinant proteins. Analytical techniques known by the skilled person in the art will be also employed to determine the structure and to check the stability of the structurally modified protein. For instance, mass spectrometry analysis, fluorescence testing and ELISA test, among many other, might be used.

By this in situ approach, the stabilized recombinant protein can be obtained directly.

This process is suitable for the stabilization of proteins comprising a large number of amino acids. This process leads to a structure-determined modified protein, the modification being in the tri-dimensional structure of the protein. It is a process for the stabilization and functional customization of proteins through the incorporation of a stable, conformation-determining amino acid in a protein sequence.”

Alternatively and pending identification, characterization and isolation of the modifying enzymatic activity that converts Phe in the recognition sequence into didehydrophenylalanine, recombinant proteins containing the recognition sequence can be produced in non-plant protein production systems and activated through incubation with the modifying enzyme. Using current state-of-art techniques recombinant proteins can be produced in prokaryotic and eukaryotic cell cultures, which based on current knowledge, do not have the enzymatic activity to convert Phe in the sequence Phe-x1-x2-Tyr into didehydrophenylalanine. After isolation of a recombinant protein containing the recognition sequence Phe-x1-x2-Tyr from a non-plant expression system it can be stored in an inactive fold. Incubation of such recombinant protein with the, currently unknown, enzymatic function that converts Phe in the recognition sequence into didehydrophenylalanine will effect a change in the fold of the recombinant protein. This would allow production, storage and distribution of proteins, like insulin or lipase, in an inactive structure followed by activation through incubation with the currently unknown modifying enzyme.

Such ex-situ approach would allow to decouple in time and space the production of recombinant proteins from their application.

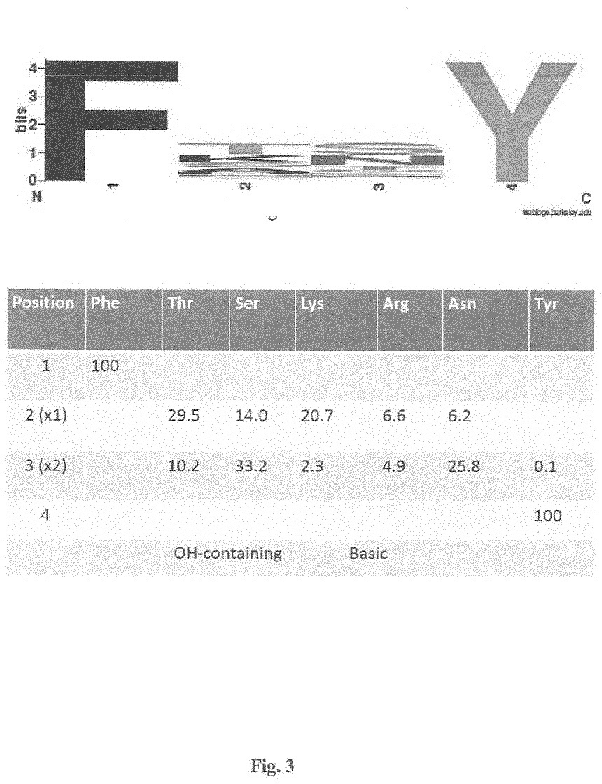

FIG. 3 shows the bioinformatic analysis of 512 recognition sequences from βPG proteins from plant species covering the entire kingdom Plantae. These sequences are obtained from the NCBI database and the analysis and graphical representation is generated with WebLOGO (https://weblogo.berkeley.edu/logo.cgi). The position 1 is Phe while at 4 Tyr makes up for 100%. The dominance of Thr, Ser, Lys, Arg and Asn at the positions 2 and 3, respectively being x1 and x2 in the annotation Phe-x1-x2-Tyr is shown. More precisely, occurrence, in percentage, of the most prevalent amino acids at the positions 1-4 in the sequence Phe-x1-x2-Tyr here defined as recognition sequence. The Phe at position 1 is converted to didehydrophenylalanine. Positions 1 and 4 are always respectively Phe and Tyr, the sum of these 5 amino acids represent 77% of the amino acids found at position 2 (x1) and 76.4% of the amino acids found at position 3 (x2). Data obtained by analysis of 512 recognition sequences from βPG proteins from plant species covering the entire kingdom Plantae. These sequences are obtained from the NCBI database and the analysis and graphical representation is generated with WebLOGO (https://weblogo.berkeley.edu/logo.cgi), as mentioned above.

The stabilized recombinant protein produced in situ or ex situ may be used in different systems as biocatalysts (e.g. production of biodiesel by lipases, biomass valorisation, lignin cleavage, etc.) or in protein therapeutics (e.g. stabilized forms of insulin, stabilized forms of antibodies, etc.).

In this recombinant protein, the presence of didehydrophenylalanine will result in a determined/stabilized fold, the exact position and structure of which is for each individual targeted protein to be determined by modelling and informatic analysis prior to the construction of the genetic construct coding for the recombinant protein. Structural constraints due to the presence of didehydrophenylalanine in an amino acid sequence are known and intensively studied (Crisma et al. J. Am. Chem. Soc. 1999, 121, 14, 3272-3278 https://doi.org/10.1021/ja9842114; Gupta et a! Biopolymers. 2011; 95(3): 161-173. doi:10.100²/_{b}ip.21561).

The use of chemical peptide synthesis, as done in Menting et al. (PNAS 2014, 111(33) E3395-E3404), to generate insulin analogues with didehydrophenylalanine on the positions 24 and 25 of the beta chain shows new functional and potentially therapeutic properties for such analogues. However the cost of chemical peptide synthesis precludes the use of this technique for producing more than milligrams of protein and the less than 100% cyclic efficiency limits its use to the production of fold-stabilized proteins with less than 100 amino acids, limitations overcome using protein synthesis capacities present in plants and plant cells as according to the invention. The structure of the insulin structurally-modified recombinant protein is identical as that disclosed in Menting et al.

Due to their selectivity in substrate and product, the applications of recombinant proteins as biocatalyst are numerous however still limited because of stability and durability issues. Approaches to overcome this are proposed (eg. Cejudo-Sanches et al. Process Biochemistry 2020, 92, 156-163 https://doi.org/10.1016/j.procbio.2020.02.026), the inclusion of didehydrophenylalanine as a result of the conversion of Phe in the sequence Phe-x1-x2-Tyr in the sequence of a recombinant protein forms an alternative means to attain such stabilization. Based on modelling and informatic analysis, the sequence Phe-x1-x2-Tyr is inserted in a genetic construct coding for the desired stabilized proteins, this genetic construct is expressed in a system inherently having the enzymatic activity that converts Phe in the sequence Phe-x-x2-Tyr into didehydrophenylalanine (based on current scientific knowledge, a plant or a plant-cell), the biocatalyst with a stabilized fold can be isolated from the expression host using current practices and applied as biocatalyst. Groups of recombinant proteins used for stabilization include lipases, proteases and nucleotide ligases.

1. A structurally-modified recombinant protein obtained by a method for producing the structurally-modified recombinant protein, comprising the steps of:
(a) generating at least one genetic construct comprising a nucleotide sequence coding for the protein comprising a recognition sequence;
(b) expressing in a host the at least one genetic construct using a vector comprising the at least one genetic construct; and using a plant-based expression system with the vector to express the protein, the plant-based expression system being a plant or a plant cells suspension;
the recognition sequence comprises a sequence Phe-x1-x2-Tyr, wherein Phe is phenylalanine, x1 and x2 are amino acid residues, and Tyr is tyrosine and the plant-based expression system has an inherent enzymatic activity which converts the phenylalanine residue of the recognition sequence into a didehydrophenylalanine residue, resulting in the structurally-modified recombinant protein, and at least one subsequent step of
(c) isolating the protein with the recognition sequence which is a part of the protein, wherein the phenylalanine has been converted to a didehydrophenylalanine from the plant-based expression system.

2. The structurally-modified recombinant protein according to claim 1, wherein x1 and x2 are polar hydroxyl-containing amino acids and/or basic amino acids.

3. The structurally-modified recombinant protein according to claim 2, wherein x1 and x2 are the hydroxyl-containing amino acids Thr and Ser, the basic amino acids Lys and Arg and the amide Asn, together accounting of from 70% to 80% of the amino acids found at the positions x1 and x2 of the sequence Phe-x1-x2-Tyr .

4. The structurally-modified recombinant protein according to claim 2, wherein x1 and x2 are the hydroxyl-containing amino acids Thr and Ser, the basic amino acids Lys and Arg and the amide Asn, together accounting of from 70% to 76% of the amino acids found at the positions x1 and x2 of the sequence Phe-x1-x2-Tyr.

5. The structurally-modified recombinant protein according to claim 2, wherein x1 and x2 are the hydroxyl-containing amino acids Thr and Ser, the basic amino acids Lys and Arg and the amide Asn, together accounting especially of from 75% to 80% of the amino acids found at the positions x1 and x2 of the sequence Phe-x1-x2-Tyr.

6. The structurally-modified recombinant protein according to claim 1, wherein the structurally-modified protein is used as a biocatalyst.

7. The structurally-modified recombinant protein according to claim 1, wherein the structurally-modified protein is used as a protein therapeutic.

8. The structurally-modified recombinant protein according to claim 1, wherein the plant-based expression system is based on at least one plant belonging to the clades of rosids.

9. The structurally-modified recombinant protein according to claim 8, wherein the rosids comprise fabids or malvids.

10. The structurally-modified recombinant protein according to claim 1, wherein the plant-based expression system is based on Medicago sativa, Arabidopsis thaliana and/or Cannabis sativa.

11. The structurally-modified recombinant protein according to claim 1, which is lipase or insulin.


SEQUENCE LISTING (6 sequences)

SEQ ID NO: 1 - protein, 22 aa, Medicago Sativa
  798..819: Beta subunit of polygalacturonase (contig 53863)
SDFSGYSEKGSSAVDAFVSYSK

SEQ ID NO: 2 - protein, 644 aa, Medicago Sativa
  Beta subunit of polygalacturonase (contig Medtr8g064530)
MKKMQKQLGHIIHTLFILLSILTVGLAINHEKEKNQNPFTPKAYVIRYWDRVIKNNLPKPSFILSKASPLSATEAATFTKHAAANTLSTKLPEFCSAAHLFCFPELTKQRPFDPSHFTVYETDQNFTFYSLGENFTTYGTHKLNGIDAFKNYSNGLYNEKHNEFARYSKISTGHNDSFKNYALNNNEGVQTFNSYGTKSMAGFGEFKAYSEESNIPNLKFTTYTASVTGREQSFVSYSDGGNSGNQSFASYGKDSIHAVNGFTQYAKNANVFKSDFSGYSEKGSSAVDAFVSYSKNLNKPENTFKNYGKGSFAAQEKFTNYRDGAGSGDDSFTSYGENSQRGVKVDFNNYGQNPDYPKSPGSETFKGYAKGADLDHKVSFKVYNGNNNTFKDYDKKGISFASYNKTTTSTNSLSDSGSLAKNMVQPGKFFREKMLKEGTVMPMPDIRDKLPPRSFLPRSILTKLPFASSKLNEMKQVFKVSENSSMNKMMVDALSECERAPSVGETKRCVGSLEDMIDFATSVLGHSVTVRSTENVNGAGKDVMVGKVKGINGGKVTESVSCHQSLFPYLLYYCHSVPNVHVYEADLLDPVSKAKINHGVAICHLDTTAWSSTHGAFMALGSAPGQIEVCHWIFENDMTWSSAD

SEQ ID NO: 3 - protein, 30 aa, Medicago Sativa
  190..219: contig Medtr8g064530
QTFNSYGTKSMAGFGEFKAYSEESNIPNLK

SEQ ID NO: 4 - protein, 42 aa, Medicago Sativa
  94..135: Beta subunit of polygalacturonase (contig 53863)
DTNVPNLRFTTYSVGVAGRQQTFTSYSEAGNAGDQSFGNYGK

SEQ ID NO: 5 - protein, 57 aa, Medicago Sativa
  192..248: Beta subunit of polygalacturonase (contig 53863)
GSLGAVEKFSNYRDQANVGADSFSSYAKDSTGGTHVDFDNYGKSFNEGTDSFKGYAK

SEQ ID NO: 6 - protein, 10 aa, Medicago Sativa
  273..282: Beta subunit of polygalacturonase (contig 53863)
KGISFAGYTK